(19) United States
(12) Patent Application Publication
(10) Pub. No.: US 2007/0197954 A1
(43) Pub. Date: Aug. 23, 2007

(54) Medical devices with enhanced ultrasonic visibilty

(76) Inventor: James Keenan, Ontario (CA)

Correspondence Address: KNOBBE MARTENS OLSON & BEAR LLP, 2040 MAIN STREET, FOURTEENTH FLOOR, IRVINE, CA, 92614, US

(21) Appl. No.: 10/549,881

(22) PCT Filed: Feb. 10, 2004
(86) PCT No.: PCT/CA04/00174

§ 371 Date: Jan. 16, 2007

Related U.S. Application Data
(63) Continuation-in-part of application No. 10/389,803, filed on Mar. 18, 2003, now abandoned.

(30) Foreign Application Priority Data
Jun. 25, 2003 (CA) 2433205

(51) Int. Cl. A61N 1/30

(52) U.S. Cl. 604/20; 604/19

(57) ABSTRACT

A medical device having enhanced ultrasonic visibility is provided. The device permits localized drug delivery, probe positioning, fluid drainage, biopsy, or ultrasound pulse delivery, through the real-time ultrasound monitoring of the needle tip position within a patient. The device permits controlled dispersion of a drug into solid tissue, the lodging of particles into solid tissue, and drug delivery into specific blood vessels. As a needle is inserted, a fluid that contrasts echogenically with the organ environment is injected into the patient. The fluid travels a brief distance before being slowed and stopped by the patient's tissue and this fluid flow will be detectable by ultrasound. The needle position during insertion will be monitored using ultrasound until it is at the desired point of action. A therapeutic drug is then delivered or a probe inserted

### FIELD OF THE INVENTION

The invention generally pertains to medical devices employing ultrasonic guidance of positioning. Such devices can be used, for example, to probe, inject therapeutic agents, drain bodily fluids, perform biopsy or to provide diagnostic imaging agents. Such devices can be used to enhance the dispersion of a therapeutic agent into solid tissue as well as to deliver a therapeutic agent into specific blood vessels. Such devices can be used to precisely position a probe within a patient in order to permit solid tumor ablation through heat, freezing, or brachytherapy.

### BACKGROUND TO THE INVENTION

Medical Rationale

Accurate, real-time knowledge of a needle tip location is an obvious requirement of a biopsy procedure. It is also desired in order to deliver drugs to a specific target site as well as to avoid puncture damage to other tissue.

Biotherapeutics, which are expected to comprise more than half of the new drugs developed in the next two decades, are often large molecules that degrade rapidly in the bloodstream and have a limited ability to cross cell membranes. Oral and intravenous delivery techniques may prove inadequate, and some biotherapeutics may require localized injection delivery.

Localized drug delivery permits a higher concentration of a therapeutic agent at the target site while minimizing side effects, as in the case of cytotoxic chemotherapy drugs. Localized delivery also results in a reduction of the required dosage amount and therefore cost, which is of benefit for applications such as gene therapy.

Intratumoral injections, such as ablation of liver tumors through alcohol injection, require precise needle positioning and fluid delivery.

Solid tumor ablation using probes is achieved through heat, using radiofrequency or microwave (RF or MW) sources, freezing (cryosurgery), or brachytherapy. Accurate positioning of the probe tip within the tumor is an obvious requirement for effective treatment.

Anti-angiogenics are drugs designed to damage tumours by attacking the blood vessels that feed them. Embolization, or clotting,, of such blood vessels is also practiced. A device that permits the delivery of a drug or material to a particular blood vessel could enhance the efficacy of such treatments.

Gel therapeutics are typically highly viscous and may be difficult to eject into a patient with standard syringes. Mechanized or motorized syringe plunger actuation would offer improved ergonomics for gel drug delivery.

Ultrasound Imaging

Ultrasound is a standard technique to image the internal body for diagnoses, and these images are usually displayed on a monitor in grey-scale. Doppler ultrasound techniques (color Doppler sonography, pulsed Doppler ultrasound, continuous wave CW Doppler, and power Doppler sonography) are typically used to measure or image blood flow. The ultrasound signal bounces off of moving blood cells and returns to the transducer, with the returning echo shifted in pitch by the Doppler effect. The moving objects can be assigned a color so that they appear in color against a grey-scale background, such as a patient's internal organs.

Doppler ultrasounds detect the motion of red blood cells, which are bioconcave discs about 7.5 micrometers in diameter and which comprise 40 to 45 percent of the blood. Color Doppler ultrasounds can detect displacements as low as microns (1 micron=0.001 millimeter) and at speeds in the 1 to 100 centimeters per second range.

Ultrasound Imaging of Needles

Smooth, thin needles are difficult to perceive in ultrasound output image unless the ultrasound pulses approach the needle at close to ninety degrees. Core biopsy needles are typically 14 to 18 gauge while needles for drug injection range from 18 to 26 gauge or beyond.

Patents to enhance the ultrasonic visibility of needle tips have been granted. One approach is to roughen or groove the needle tip but this may increase the trauma of needle insertion.

Other approaches to enhance ultrasound visibility include: producing bubbles at the needle tip to better reflect ultrasound, mounting miniature transducers at the needle tip, vibrating a solid stylet carried coaxially within a hollow biopsy needle, reciprocating a stylet longitudinally using a solenoid coil in the syringe, and using transducers to generate a longitudinal oscillation of a fluid column coupled to the needle tip. A difficulty encountered by some of these approaches is that motion was not confined to the needle tip and the Doppler ultrasound colored the entire needle. An invention that featured a loudspeaker connected to a hollow stylet was successful in displaying the needle tip as a color beacon regardless of the angle of incidence of the Doppler beam, but tissue material could block the needle during insertion and stop the color signal at the tip.

Syringes and Syringe Pumps

Injecting fluid into a patient with sufficient speed and duration to be detectable by ultrasound can be accomplished with a standard syringe and the force of a person's thumb. However, it is difficult to consistently control the fluid flow manually in order to precisely locate the position of the needle tip using ultrasound.

Double-barreled syringe pumps are commercially available for medical uses and for mixing epoxy. These devices do not pertain to enhanced ultrasonic visibility of a needle.

Microprocessor controlled, automated syringe pumps are established technology. They may be used to intravenously deliver controlled volumes of a drug, in a time-released manner, to a patient. Some pumps incorporate occlusion detection means. They are not configured to eject fluid pulses during needle insertion in order to enhance the ultrasonic visibility of a needle. Commercial manufacturers include Fisher Scientific for laboratory applications, insulin pumps from Animas Corporation, and intravenous infusion pumps from Baxter.

Fluid Pressure Monitoring of Medical Devices

Using pressure to precisely locate the distal end of a delivery tube was disclosed in U.S. Pat. No. 6,251,079, by Gambale, et al, in ‘Transthoracic drug delivery device’. However, that invention comprised a pressure sensing tube mounted in parallel to a drug delivery tube to provide transthoracic drug delivery, in particular for therapeutic substances to be ejected into the myocardium.

Fluid Conditioning of Tissue

Needlelesg injection devices force liquids through the skin at speeds up to 400 meters/second using compressed gas. Potentially, fluid pulses, with precisely controlled flow rates and flow volumes, could condition tissue prior to injecting a therapeutic agent and enhance the dispersion of the drug.

Ultrasound Rupturing of Microspheres

Acoustically active drug delivery systems consist of gas filled microspheres that, under external ultrasound, rupture to release a therapeutic compound in a specific region of the body. Acoustically activated drug delivery systems include microspheres, microbubbles, drug impregnated microsponges, injectable nanoparticles such as vesicles, micelles, and liposomes, and other drug carrying particles that permit acoustic activation of therapeutic agents. Tachibana et al, Fukuoka University School of Medicine, describe a method in ‘The Use of Ultrasound for Drug Delivery’ [Echocardiography—Jnl Cardiovascular Ultrasound & Allied Techniques 18 (4), 323-328.doi: 10.1046/j.1540-8175.2001.00323.x]: “Recent studies have shown that nonthermal ultrasound energy could be applied for targeting or controlling drug release . . . [of ] echo contrast microbubbles . . . used to carry and release genes to various tissues and lesions.”

Although designed for intravenous delivery, the microsphere approach may show enhanced efficacy if delivered via needle with a transducer mounted in the syringe delivering the rupturing ultrasound pulse down through the needle.

Time Reversed Acoustics Therapy

The precise focusing of therapeutic ultrasound to an internal point of interest, such as a tumor, using transducers contacting a patient's skin has proven to be very difficult. A time reversed acoustics therapy under development consists of:
positioning an ultrasound source within a tumor
emitting ultrasound and tracking this emission using an external array contacting the patient
withdrawing internal ultrasound
applying therapeutic ultrasound as required from the external array, using the tracked emission pattern to precisely focus the ultrasound on the point of interest

### SUMMARY OF THE INVENTION

A medical device with enhanced ultrasonic visibility is provided. The ultrasonically enhanced device comprises, a fluid container having a discharge end, a fluid discharge means disposed in connection with the fluid container so as to define a fluid retaining reservoir, the discharge means for applying a selected pressure to a fluid in the fluid retaining reservoir for ejecting said fluid from the reservoir through the discharge end, a first conduit having an entrance end and an exit end and defining a first passage therebetween, the entrance end disposed at the discharge end of the fluid container, the first passage in communication with the reservoir; a needle having a connector end and a distal tip and defining a needle passage therebetween, the connector end disposed at the exit end of the first conduit, the needle passage in communication with the first passage; a fluid supply means operatively connected to the fluid discharge means for selectively applying the selected pressure to the fluid, whereby the selected pressure ejects the fluid through the discharge end of the fluid container and travels a first flow path through the first passage and through the needle passage, for ejection at the distal tip at a fluid flow rate selected for detection by ultrasound. The fluid may be an echogenic fluid, such as saline alone or in combination with another therapeutic agent.

The medical device may be housed completely or in part into a handheld assembly. The fluid container may be in the form of a syringe and the fluid discharge means may be in the form of a plunger for the syringe.

As a needle is inserted to a depth within a patient, an ultrasound imaging system may no longer be able to detect and display it. The invention permits a fluid to be injected into the patient as the needle is inserted. The fluid will travel a brief distance before being slowed and stopped by the patient's tissue, and this speed and travel distance will be of sufficient magnitude as to be detectable by an ultrasound imaging system. The fluid flow or pulse will highlight the position of the needle tip under real-time ultrasound guidance.

The position of the needle tip may be monitored during insertion until said tip is positioned at the desired point of action, such as a particular organ or a cancer tumor. In certain embodiments, an adaptor releasably couples the needle to the device. Once positioned at the desired point of action, the needle may be detached and substituted with another needle or a probe, or alternatively, a probe may be provided within the needle passage. Various different probes may be used for applying a variety of therapy.

The medical device may also include a port connector; a second conduit having a second entrance end and a second exit end and defining a second passage therebetween, the second exit end disposed at the port connector; a second connector disposed at the second entrance end for connection for the second entrance to a selected medical, component, wherein the port connector is disposed at a selected portion of the first conduit or at the valve member for permitting communication between the second passage and the first passage. A variety of medical components may be selected, including a second fluid container in the form of a second syringe and a second fluid discharge means in the form of a second plunger for the second syringe. The second syringe may be used to deliver a therapeutic agent. Alternatively, the medical component may be a vacuum source for use in a biopsy procedure.

At the desired point of action, different embodiments of the device may be used to:
deliver a second fluid, such as a therapeutic drug
deliver a plurality of fluids, such as a two-part therapeutic agent to be intermixed in vivo
aspirate tissue for biopsy or drain bodily fluids using a vacuum pump
ablate tumors using a probe or probes
position a flexible fluid conduit to permit repeat dose, localized drug delivery
control the dispersion of a therapeutic agent into solid tissue
lodge particles, such as drug eluting or radiolabelled particles, into solid tissue
deliver therapeutic agents into specific blood vessels
enhance the dispersion of a therapeutic agent using ultrasound pulses transmitted through the needle
rupture drug eluting microspheres in vivo using ultrasound pulses transmitted through the needle
position an ultrasound source into a point of interest, such as a tumor, in order to permit time reversed acoustics therapy

During needle insertion, the echogenic fluid that highlights the position of the needle tip may be pumped continuously or intermittently. In the mechanized embodiment of the invention this is accomplished using manual controls. In the electromechanical embodiment this may be accomplished using manual controls, or by programmed pulses using a processor.

The position of the needle tip may be monitored through an ultrasound display and the fluid flow rate may be adjusted. This will vary the volume of space detectable by the ultrasound so as to maintain a properly defined image of the position of the needle tip.

The invention also incorporates ultrasonic systems and methods of using such ultrasonically enhanced devices.

It is to be appreciated that reference to a “device” of the present invention may be understood to include an “apparatus” or “assembly”, which may be incorporated into systems with suitable adaptations.

It is also to be appreciated that the devices of the present invention may be used in a variety of applications, including medical diagnosis, treatment, surgery, and the like, and also may be used in a similar fashion in veterinary applications with suitable modifications.

The foregoing summarizes the principal features of the invention and some of its optional aspects. The invention may be further understood by the description of the preferred embodiments, in conjunction with the drawings, which now follow.

### BRIEF DESCRIPTION OF THE DRAWINGS

The accompanying drawings illustrate presently preferred embodiments of the invention and, together with the description that follows, serve to explain the principles of the invention.

FIG. 1 depicts an electromechanical embodiment of the invention being used to deliver drugs.

FIG. 2 depicts an electromechanical embodiment of the invention being used to perform biopsy.

FIG. 3 depicts a side view of the handheld assembly with the therapeutic agent (not shown) and an echogenic fluid contained in syringes with plungers.

FIG. 3A depicts a top view of the fluid flow and mechanical drive of the handheld assembly in the embodiment with the therapeutic agent and echogenic fluid contained in syringes.

FIG. 4 depicts an isometric view of the switch mechanism and mechanical drive portion of the handheld assembly, configured to deliver drugs.

FIGS. 5A and 5B depict top and, side views of an embodiment of the invention using a mechanical mechanism to transfer the fluid.

FIG. 6 depicts an embodiment of the invention consisting of a handheld adapter connected to a commercial intravenous infusion pump.

FIG. 7 depicts an embodiment of the invention wherein an ultrasound source is incorporated in the handheld assembly in order to enable ultrasound delivery to the patient through the needle.

FIG. 8 depicts an embodiment of the invention with vessels for three fluids: an echogenic fluid, and two therapeutic agents.

FIGS. 9A and 9B depict an embodiment of the invention with tumor ablation probes incorporated into the syringe pump.

### DETAILED DESCRIPTION OF PREFERRED EMBODIMENTS

Reference will now be made in detail to various suitable embodiments of the invention as illustrated in the accompanying drawings. It will be understood that this description is exemplary and is to assist in understanding the invention and the principles of operation.

Devices of the present invention includes means for providing an echogenic fluid from a needle, and analysis thereof, to enhance the ultrasonic visibility of the needle tip. The device may be comprised of a handheld assembly or of a system, comprised of a handheld assembly connected to other components such as fluid vessels, power sources, and meters.

The term needle is intended to include any hollow, slender instrument that may be manipulated to puncture or be inserted or otherwise probe tissues, organs, cavities, or the like. The needle may be used to introduce material into or remove material from a patient or to perform other therapeutic or diagnostic functions. The term needle is intended to include-rods or wire-like medical instruments, cannulas, probes, tubes and lumens, stylets, and the like. The patient may be any suitable animal, including humans.

Fluid is defined to mean any suitable liquid, suspension, or gas.

The fluid supply means may be a syringe pump, variable speed fluid transfer pump, peristaltic pump, or other means to pump fluids.

The fluid supply means may be driven by mechanical means such as compression or extension springs, or other mechanical methods, by electro-mechanical means such as an electric motor, solenoid drive, or other electro-mechanical means, or by pneumatic or hydraulic means.

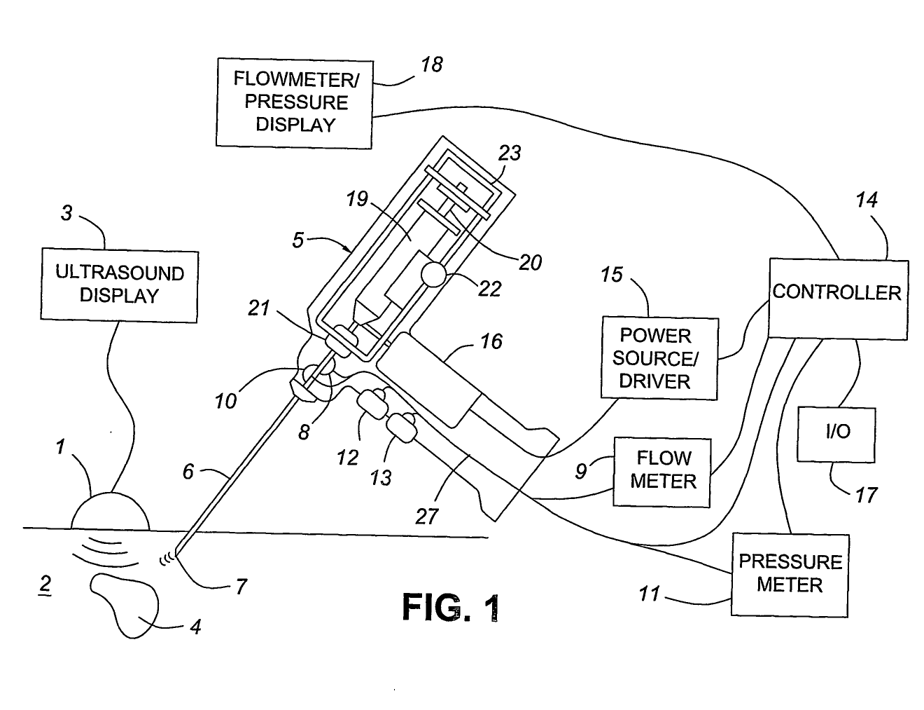

An electro-mechanical embodiment of the device comprised of a handheld assembly, needle, needle adapter, syringes to contain two different fluids, fluid conduit, fluid pump, controls, pressure sensor, flow sensor, fluid switching mechanism, valve, electric stepper motor, a drive shaft, and linkages, is shown in FIGS. 1, 2, 3, 3A, and 4.

FIG. 1 depicts the device being used to perform localized drug delivery at a depth within a patient.

An ultrasound transducer (1) transmits and receives pulses in order to image the interior of a patient (2) on an ultrasound display (3). The handheld assembly (5) is used to insert a needle (6) into the patient towards the desired point of action (4), an organ, tumor, etcetera. Fluid is ejected from the distal tip of the needle (7) at sufficient speed, and for sufficient travel distance, as to be detectable by the ultrasound.

A wide range of fluid speed and travel distance could be detectable by ultrasound: 1 cm/sec up to 100 m/sec and 10 microns up to 2 centimeters. Greater ranges may also be detectable.

Ultrasound equipment can be used to image blood flow and sonographers are often experienced in doing so. Therefore, it may be advantageous to set the echogenic fluid flow rate between 30 and 300 cm/sec, corresponding to the flow rates typically seen in human blood vessels.

Real time monitoring of the needle position may be done with standard or Doppler ultrasound. If Doppler is chosen, the patient's internal organs may be displayed in grey-scale while a distinct color is assigned to the ejected fluid flow at the needle tip.

A flow meter sensor (8) mounted on the distal end of the handheld assembly (5) is connected to the flow meter (9). A pressure sensor (10) mounted on the distal end of the handheld assembly (5) is connected to the pressure meter (I1). Trigger controls (12) and (13) may be used to switch the flow on/off and to adjust the flow rate.

If too high a fluid flow rate is ejected during the needle insertion it could disrupt tissue and the fluid distribution could be unpredictable. The fluid could flow for centimeters in multiple directions and too large a volume of space would be detected by the ultrasound to permit precise monitoring of the needle tip. Therefore, real time flow rate adjustments may be required in order to contain the zone of flowing fluid to a small volume of space in proximity to the needle tip.

A transducer to sense the position and speed of the syringe plungers, and connected to the controller, could also be used to sense the fluid flow rate.

The controller (14) is a microprocessor connected via a wire wrap cable (27), to the manual controls, power source and driver (15), flow meter (9), pressure meter (11), input/output (17), and the flow meter/pump pressure display (18). The controller input/output (17) permits the entry of commands to specify pulsed flow etcetera.

The power source and driver (15) drives the syringe pump motor (16), which is linked to a drive shaft (not shown), that actuates the plunger (20) for the syringe containing the echogenic fluid (19). Once the needle tip (7) is ositioned at the desired point of action (4), the flow of Fluid 1 and may be stopped and Fluid 2, the therapeutic agent (syringe not shown) be injected into the patient (2).

The motor RPM range, gear ratio between the motor link, drive shaft and syringe plunger actuator links, the motor driver card, and the automatic controls may be specified to enable minute, real-time adjustments to the flow rate and to control the delivered volume of the therapeutic agent.

The fluid flow is switched using a manual switching mechanism (23) connected to a push button (22). The switching mechanism (23) simultaneously engages/disengages the syringe plunger actuators from one syringe to the other as well as switching the fluid valve (21) from one syringe to the other.

There are a number of options for Fluid 1, the echogenic fluid to be ejected during the needle insertion to highlight the position-of the needle tip. The key requirements are that Fluid 1 be relatively biologically harmless (such as sterile saline) and contrast, in an echogenic sense, with the surrounding tissue environment. The fluid may be more or less echogenic than the tissue environment.

Fluid 1 must have minimal adverse effect on Fluid 2, the therapeutic agent, as the needle and fluid conduits will not be flushed between injections of the two fluids. Fluid 1 could contain drugs that aided the efficacy of the therapeutic agent, such as a drug to prevent infection or to aid or to combat the migration of the therapeutic agent. It could also contain a chemical additive to decrease its' viscosity. Fluid 1 could be the patient's own blood, reused as per a transfusion, an echogenic gas, or sterile water.

Alternatively, carbon dioxide gas may be a suitable, echogenic fluid as it disperses in the body and is notably echogenic. It could be, delivered through a liquid filled needle and into a patient in the form of small gas bubbles.

Fluid 2, the therapeutic agent delivered at the point of action, could be: a liquid drug, solid drug particles suspended in a fluid, drug eluting microspheres suspended in a fluid, or other therapeutic agents that can be delivered under pressure through a needle. A small quantity of the therapeutic agent, 0.2 to 1.0 ml, may be delivered.

The device can be used to control the dispersion pattern of a delivered drug. Once the needle tip is positioned at the point of action, the echogenic fluid may be pulsed, repeatedly and at a variety of flow rates if necessary, and the fluid dispersion pattern monitored. The flow rates of these preliminary fluid pulses can be high enough to condition the tissue at the point of action, which may benefit the drug dispersion. Once the dispersion pattern is satisfactory, a second fluid, the therapeutic agent, can then be delivered.

The device may be used to pulse particles in suspension into solid tissue in order to lodge the particles into the tissue and permit localized treatment over prolonged periods of time. These particles may be drug eluting, drug-filled microspheres, biodegradable particles, radiolabelled glass frits, radiolabelled metallic, ceramic, or plastic, or other solid therapeutic agents in suspension.

The device may be used to deliver a therapeutic agent into a specific blood vessel using a fluid pressure meter mounted in the handheld assembly. The pressure required to maintain a constant flow rate will vary as the back pressure varies, and this back pressure may drop if the needle tip pierces a blood vessel wall and the echogenic fluid is ejected directly into an artery or vein. Therefore, by monitoring the pressure and rate of change of the pressure, the needle may be positioned to deliver drugs directly into a particular blood vessel. An auditory or visual alarm could be incorporated into the system to signal when the pump pressure has dropped and the needle tip has pierced a blood vessel wall.

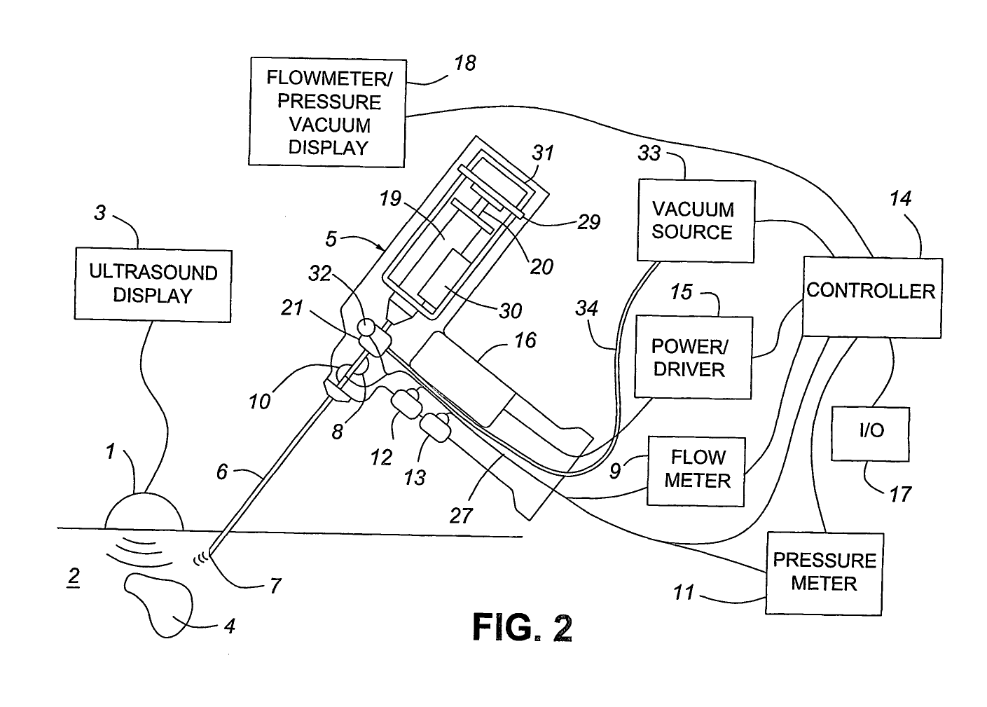

FIG. 2 depicts the device being used to perform biopsy at a depth within a patient.

An ultrasound transducer (1) transmits and receives pulses in order to image the interior of a patient (2) on an ultrasound display (3). The handheld assembly (5) is used to insert a needle (6) into the patient towards the desired point of action (4), an organ, tumor, etcetera.

Fluid is ejected out of the distal tip of the needle (7) at sufficient speed; and for sufficient travel distance, as to be detectable by the ultrasound.

A flow meter sensor (8) mounted on the distal end of the handheld assembly (5) is connected to the flow meter (9). A pressure sensor (10) mounted on the distal end of the handheld assembly (5) is connected to the pressure meter (11). Trigger controls (12) and (13) may be used to switch the flow on/off and to adjust the flow rate.

The controller (14) is a microprocessor connected via a wire wrap cable (27), to the manual controls, power source and driver (15), flow meter (9), pressure meter (11), input/output (17), vacuum source (33), valve (32), and the flow meter/pump pressure/vacuum display (18). The controller input/output (17) permits the entering of commands to specify pulsed flow etcetera. The vacuum source (33) is connected to the handheld assembly (5) with a vacuum line (34).

The power source and driver (15) drives the syringe pump motor (16), which is linked to a drive shaft (not shown). The drive shaft drives the plunger actuator (29), which slides, along support rods (31) to actuate the plunger (20) for the syringe containing the echogenic fluid (19).

Once the needle tip (7) is positioned at the desired point of action (4), the fluid flow may be stopped and the valve (32) closed. The vacuum source (33) is then used to aspirate tissue for biopsy.

A stylet may also be used with the needle to perform biopsy.

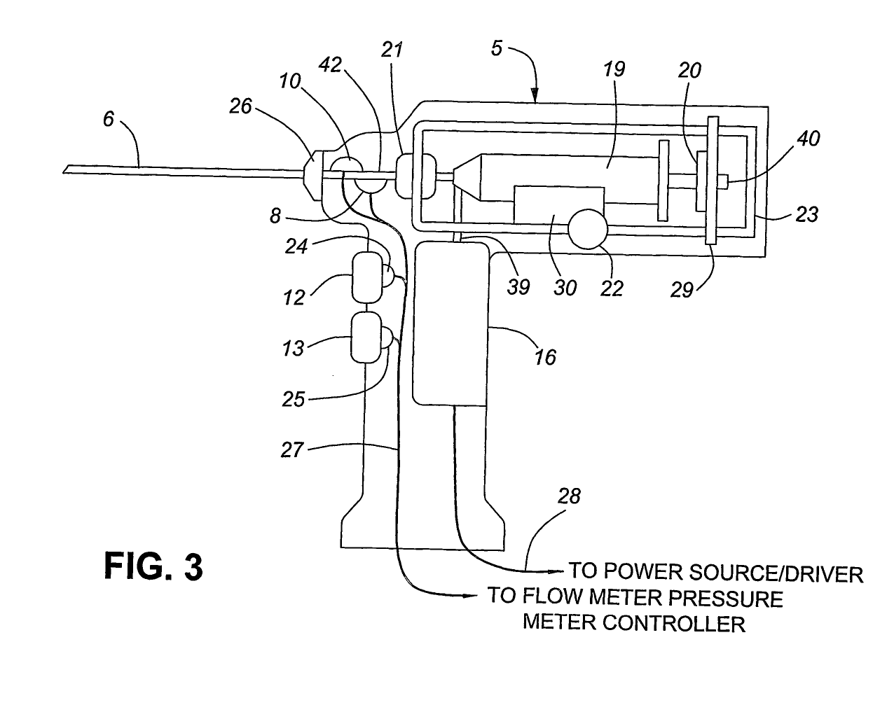

FIG. 3 depicts the handheld assembly of the device configured to deliver drugs.

A handheld assembly (5) with a needle adaptor (26) to hold a needle (6) for injecting drugs within a patient is shown. A sensor (8) detects the fluid flow rate. A pressure sensor (10) detects the fluid pressure. A top trigger control (12) with a position sensor (24) is used to set the flow rate and a lower trigger (13) and switch (25) is used to switch the flow on and off. The flow sensor (8), pressure sensor (10), top trigger position sensor (24), and lower trigger switch (25) are connected via a wire wrap cable (27), which runs out to the flow meter, pressure meter, and controller.

The power source and driver card (not shown) is connected via wire (28) to the syringe pump motor (16), which is mechanically linked (39) to a drive shaft (not shown). Alternatively, the pump motor may be battery driven (not shown). The drive shaft is linked to the plunger actuator (29) which slides along the horizontal support rods of the switching mechanism (23) to actuate the plunger (20) for the syringe containing the echogenic fluid (19). This syringe (19) is fastened to the switching mechanism (23) through an adjustable syringe clamp (36).

During insertion of the needle (6) into the patient, the plunger (20) is actuated, and Fluid 1 flows from the syringe, through a fluid valve, (21), a fluid conduit (42), and through the needle (6) where it is injected into the patient.

Once the needle tip is positioned at the desired point of action the flow of Fluid 1, the echogenic fluid, is stopped to permit flow from Fluid 2, the therapeutic agent, (syringe not shown). The fluid flow is switched by actuating a push button (22) connected to the switching mechanism (23). The switching mechanism (23) simultaneously engages/disengages the syringe plunger actuators from one syringe to the other as well as switching the fluid valve (21) from one syringe to the other.

FIG. 3A depicts a top view of fluid flow and mechanical drive portion of the handheld assembly, configured to deliver drugs.

The syringe pump motor (16) is mechanically linked (39), to a drive shaft (37), which is supported by two bearings (38). The drive shaft is mechanically linked (40) to either syringe plunger actuator (29), which slide parallel to the drive shaft along the horizontal support rods of the switching mechanism (not shown). The plunger actuators (29) drive the plunger (20) for the Fluid 1 syringe (19) or the plunger (36) for the Fluid 2 syringe (35). The syringes are moved perpendicular to the drive shaft axis by the switching mechanism (not shown) in order for either mechanical link (40) to be engaged to the drive shaft. The syringes (19) and (35) are fastened to the switching mechanism (not shown) through a pair of adjustable syringe clamps (30).

Fluid flows from either syringe through flexible fluid conduit (42), to a valve (21), and through the needle adapter (26) to the needle (6). The pressure sensor (10) and flow sensor (not shown) monitor the flow at the distal end of the handheld assembly (housing not shown).

Once the needle tip is positioned at the desired point of action the flow of Fluid 1, the echogenic fluid, (19) is stopped to permit flow from Fluid 2, the therapeutic agent, (35). The fluid flow is switched by actuating a push button (22) connected to the switching mechanism (not shown). The switching mechanism (not shown) moves the syringes perpendicular to the drive shaft to simultaneously engage/disengage the links (40) to the syringe plunger actuators (29) and to switch the fluid flow through the valve (21) with a valve actuator (41).

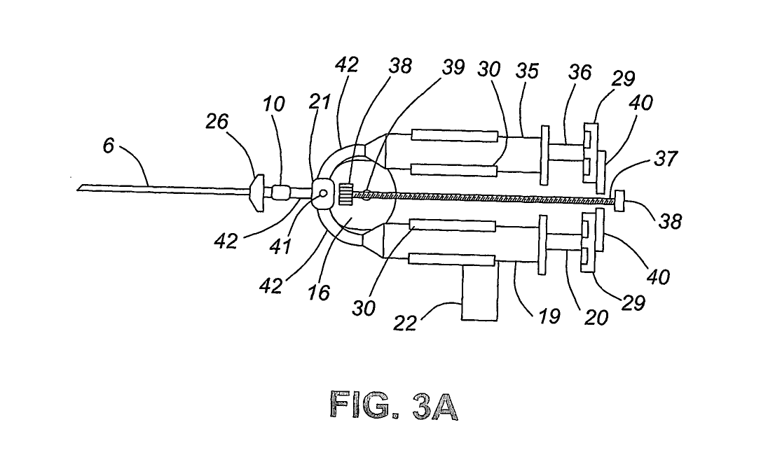

FIG. 4 depicts an isometric view of the switch mechanism and mechanical drive portion of the handheld assembly, configured to deliver drugs.

The syringe pump motor (16) is mechanically linked (39), to a drive shaft (37), which is supported by two bearings (38). The drive shaft is mechanically linked (40) to a syringe plunger actuator (29), which slides parallel to the drive shaft on the horizontal support rods of the switching mechanism (23), to actuate the syringe plunger (not shown). The syringe (not shown) is fastened to the switching mechanism (23) through an adjustable syringe clamp (30). Only one of the two sets of plunger actuators (29), links (40), and syringe clamps (30) are depicted in FIG. 4.

The fluid flow is switched by actuating a push button (22) connected to the switching mechanism (23). The switching mechanism (23) moves the syringes perpendicular to the drive shaft to engage/disengage the link (40) between the syringe plunger actuator (29) and the drive shaft (37). The switching mechanism (23) also simultaneously switches the fluid flow through the valve (not shown) with a valve actuator (41).

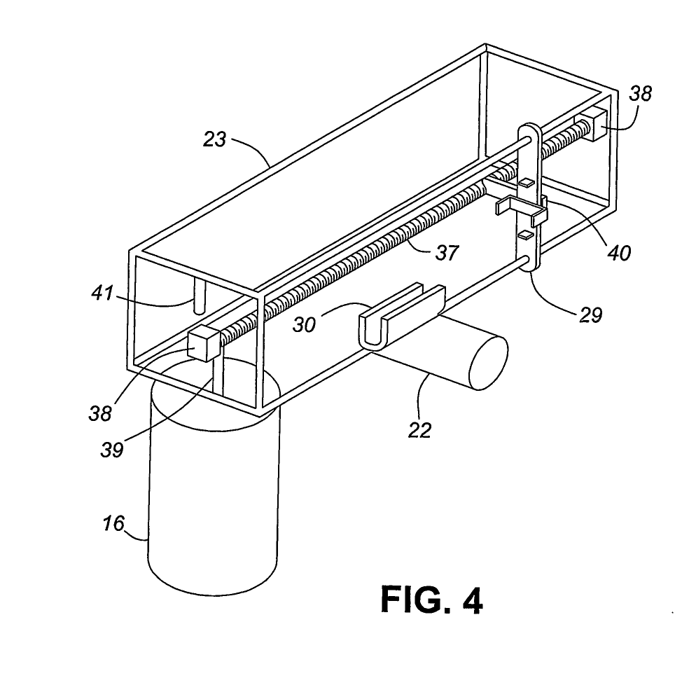

FIG. 5A and 5B depict top and side views of an embodiment of the invention using a mechanical mechanism to drive the fluid transfer.

A handheld assembly (5) with a needle adaptor (26) to hold a needle (6) for injecting drugs to a depth within a patient is shown. A pressure sensor (10) may be used to detect the fluid pressure. A top trigger control (12) is linked (not shown) to a mechanism (43) which pulses fluid from the Fluid 1 syringe (19). A lower trigger (13) is linked to a duplicate mechanism (43) which pulses fluid from the Fluid 2 syringe (35).

The mechanisms (43) consist of syringe plunger actuators (29), which clamp to the syringe plungers (20) and (36), drive springs (44), and control knobs (45) to adjust the pre-load tension of the springs (44). Such adjustment will vary the fluid flow of each pulse. The syringes (19) and (35) are fastened to the assembly (5) through a pair of adjustable syringe clamps (30).

Fluid flows from either syringe through conduit (42) and through the needle adapter (26) to the needle (6).

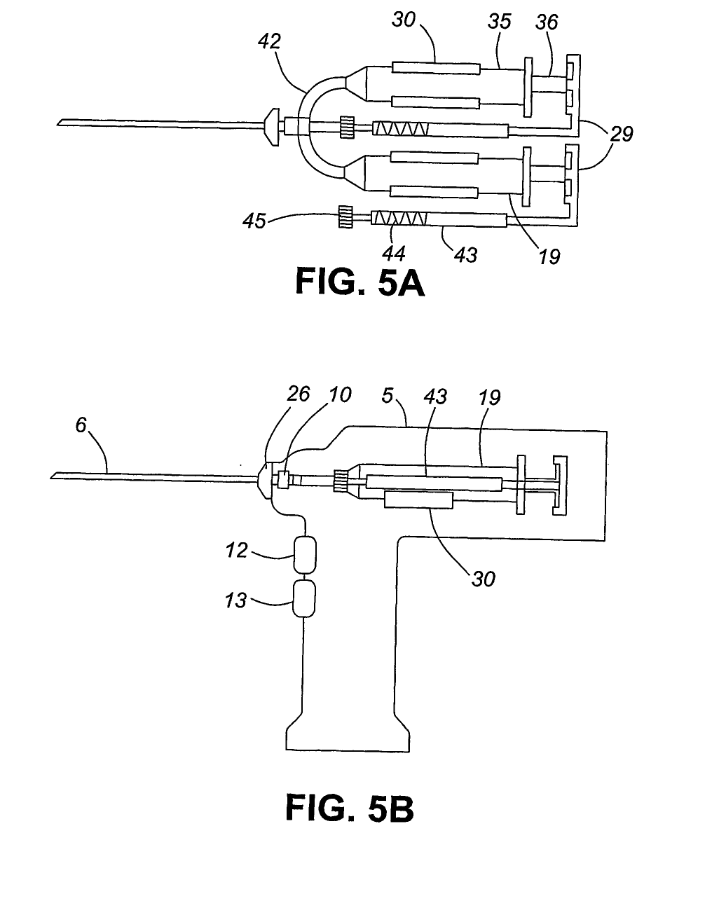

FIG. 6 depicts an embodiment of the invention, consisting of a handheld adapter connected to a commercial intravenous infusion pump.

A handheld assembly (5) with a needle adaptor (26) to hold a needle (6) for injecting drugs to a depth within a patient is shown. A pressure sensor (10) detects the fluid pressure. A trigger control (12) and switch (24) is used to switch the flow on and off. A flow adjustment knob (48) and sensor (not depicted) are used to vary the flow rate. The pressure sensor (10), flow adjustment sensor, and trigger switch (25) are connected via a wire wrap cable (27) to an electrical port (47), such as an RS232 port, on the commercial infusion pump (46).

The commercial infusion pump (46), such as a Baxter AS50, drives the fluid from the Fluid 1 syringe (19) through flexible fluid conduit (42) to the handheld assembly (5).

When the needle (6) is positioned at the desired point of action within a patient, fluid is delivered from the Fluid 2 syringe (35). The Fluid 2 syringe (35) may be mounted to the handheld assembly (5) or, as depicted in FIG. 6, the Fluid 2 syringe (35) may be held separately and manually actuated. The Fluid 2 syringe needle (49) pierces a port (50) in the handheld assembly and the fluid is ejected out of the syringe (35) and down through the handheld assembly needle (6) into the patient.

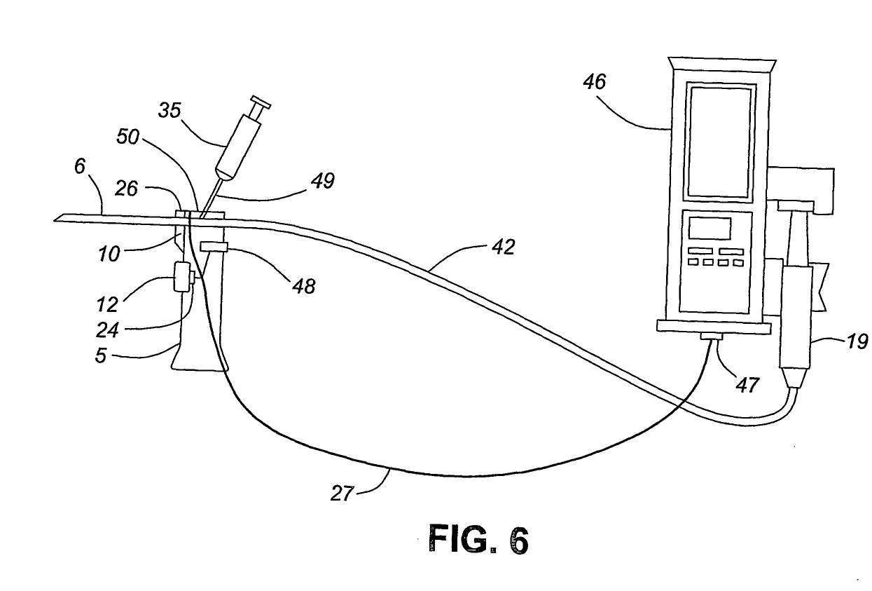

FIG. 7 depicts an embodiment of the invention wherein an ultrasound source is incorporated in the handheld assembly in order to enable ultrasound pulses to be delivered to the patient through the needle.

This will permit a more controllable, consistent ultrasound pulse to be delivered to the patient, independent of needle insertion depth, density of tissue, and other variables, than an ultrasound applied via a transducer placed on the patient's skin. The utility of such an ultrasound pulse may be:
to activate the pharmacological activity of a therapeutic agent, such as enhancing drug transport through tissues and across cell membranes, and, or
to condition the patient's tissue with ultrasound pulses in order to improve a therapeutic agent's dispersion and efficacy, and, or
to create a hyperthermic condition that can enhance the destruction of diseased tissue such as cancerous tissue, and, or
to use the device to rupture drug-eluting microspheres immediately after administration.

A handheld assembly (not shown) with a needle adaptor (26) to hold a needle (6) for injecting drugs to a depth within a patient is shown. Fluid may be pulsed from the Fluid 1 syringe (19) or the Fluid 2 syringe (35).

Fluid flows from either syringe through conduit (42) and through the needle adapter (26) to the needle (6).

A transducer probe (51), or multi-transducer array (not depicted), is mounted in contact with the fluid conduit (42) and produces ultrasound energy that is transferred down through the needle (6) into the patient. The transducer or array is connected to a controller and power source (not depicted). The controller may enable adjustment of frequency, duration, mode, power, and other parameters of the ultrasound pulses, and may or may not be connected to a display.

Alternatively, a transducer probe (51), or multi-transducer array (not depicted), could be mounted in contact with the fluid on a standard, manually actuated syringe (not depicted) to produce ultrasound energy that is transferred down through a needle into the patient.

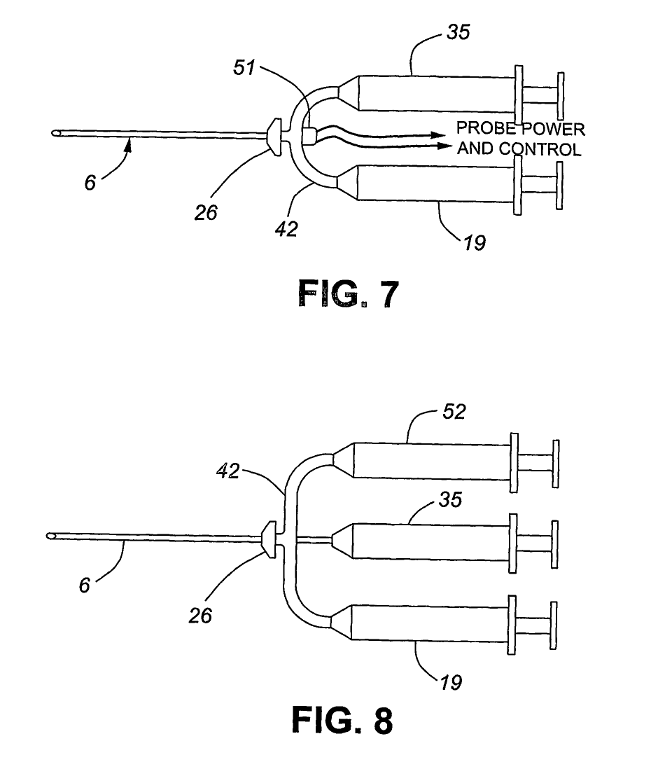

FIG. 8 depicts an embodiment of the invention with vessels for three fluids: an echogenic fluid (19), and two therapeutic agents (35) and (52). The syringe pump actuators may be used to supply fluid from any single vessel or from two or three vessels simultaneously, through the needle adapter (26) and to the needle (6).

The utility of such a device is to deliver therapeutic agents comprised of two solutions that, in order to be effective, must be intermixed immediately before administration, or in some instances intermixed in vivo in the patient.

Tumor ablation via probe could also be accomplished using two different embodiments. Once the needle has been precisely positioned, the handheld assembly may be decoupled from the needle, and a probe or probes inserted down through the needle. The probes can then be used to ablate the tumor through heat, with RF or WM energy, cryosurgical freezing, or through brachytherapy using a rod with a radioactive source at the tip.

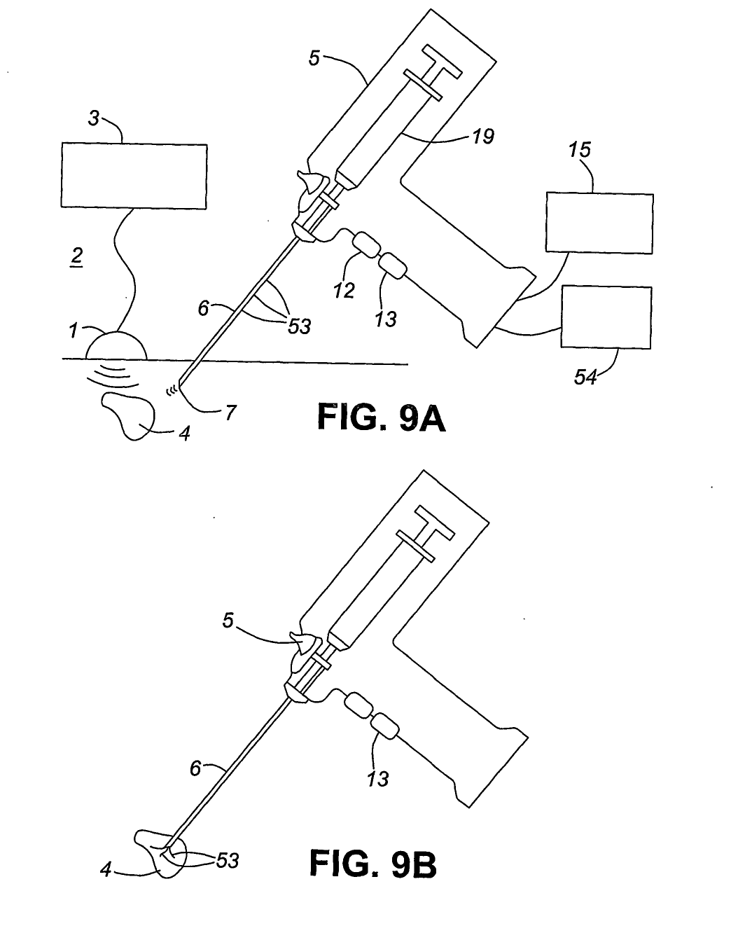

An embodiment of the invention, as depicted in FIGS. 9A and 9B, incorporates tumor ablation probes within the handheld assembly. This permits tumor ablation without the need for decoupling the needle from the handheld assembly and inserting a separate tumor ablation probe device down through the needle.

FIG. 9A depicts the invention being used to position a needle under real-time ultrasound guidance.

An ultrasound transducer (1) transmits and receives pulses in order to image the interior of a patient (2) on an ultrasound display (3). The handheld assembly (5) is used to insert a needle (6) into the patient towards the desired point of action (4), such as a solid tumor.

Fluid 1, the echogenic fluid, (19), is ejected from the distal tip of the needle (7) at sufficient speed and for sufficient travel distance as to be detectable by the ultrasound.

Radiofrequency probes (53) inside the needle (6) are connected through a sealed needle adaptor (26) to the RF control (54) and power source (15).

Trigger controls (12) and (13) may be used to adjust the flow of Fluid 1, the echogenic fluid, (19) and the RF power.

FIG. 9B depicts the invention deploying probes in order to ablate a solid tumor.

Once the needle (6) is positioned at the desired point of action (4), probes (53) are deployed within the patient's tissue using a sliding mechanism (55). The trigger control (13) adjusts the RF power in order to ablate the solid tumor.

Upon ablation of the tumor, the probes (53) may be withdrawn back into the needle (6) for withdrawal of the device.

Alternatively, repeat dose, localized drug delivery could be accomplished. Once the needle has been precisely positioned, the handheld assembly may be decoupled from the needle, and a flexible, sterile, fluid conduit inserted down through the needle using a rod. Once the fluid conduit is positioned at the point of interest, the needle and rod may be withdrawn. Repeat drug doses are then delivered through the conduit, which may be a PortaCath™, Hickman line, PICC or other type of flexible conduit for drug delivery.

The various embodiments of the device may be fitted with:
a variety of needle sizes through a leak-proof adapter, such as a threaded adapter
a variety of needle tip geometries including a standard open end, an angled open end, or a closed end with slots running along the side of the needle tip, or combinations of geometries
multiple lumen needle
a stylet incorporated into a multiple lumen for biopsy or fluid drainage use; the stylet would prevent tissue ingress into the lumen intended to aspirate tissue, while the lumen for ejecting the echogenic fluid remained open
a variety of fluid vessels, which may be held in adjustable clamps and connected to flexible fluid conduits using leak proof fittings
a removable cover for the injectate-contacting components, to permit ease of component changing for each patient procedure
a transparent cover and/or opening to enable visual monitoring of the fluid vessels and/or conduits

### CONCLUSION

A device to locally inject drugs, position probes, drain bodily fluids, perform biopsy, or apply ultrasound pulses under real-time ultrasound imaging of the needle position within a patient, is disclosed. The device may permit controlled dispersion of a drug into solid tissue as well as delivery into specific blood vessels.

The device is comprised of a handheld assembly or system with a needle, needle adapter, fluid vessels, and means to pump the fluid. It may include flow controls, a pressure sensor, flow sensor, fluid switch mechanism, and valve.

The handheld assembly may be connected to a pressure meter, flow meter, controller, controller I/O, display, and power source.

As the needle is inserted, the first fluid, a fluid to contrast echogenicly with the organ environment, is injected into the patient. The fluid travels a brief distance before being slowed and stopped by the patient's tissue. This speed and travel distance will be of sufficient magnitude as to be detectable by ultrasound.

The position of the needle tip will be monitored during insertion until said tip is positioned at the desired point of action, for instance a particular organ or a cancer tumor.

The second fluid or fluids, such as a therapeutic drug, is then delivered. Alternatively, a vacuum pump could then be used to aspirate tissue for biopsy or fluid for drainage.

Alternatively, a probe could then be inserted through the needle in order to ablate solid tumors using heat, freezing, brachytherapy or other means.

During needle insertion, the first fluid may be pumped continuously or intermittently using the manual controls, or pulsed using the processor. The needle tip position will be monitored through an ultrasound display and the fluid flow rate may be adjusted. This will vary the volume of space detectable by the ultrasound so as to maintain a properly defined image of the needle tip.

The device may be used to deliver ultrasound, down through the needle and into the patient, using a transducer or transducer array mounted in the handheld assembly. This will permit the acoustical activation of drug filled particles and other uses.

The device can also be used to control the dispersion pattern of a delivered drug. Once the needle tip is positioned at the point of action, the echogenic fluid can be pulsed, repeatedly and at a variety of flow rates if necessary, and the fluid dispersion monitored. Once this is satisfactory, the second fluid, the therapeutic agent, can then be delivered.

The device can also be used to lodge particles into solid tissue. Once the needle tip is positioned at the point of action, the flow rate can be adjusted to a sufficient magnitude in order to eject a suspension and lodge the particles in solid tissue.

The device may display the set flow rate, fluid pressure, and the rate of change of the pressure.

The pressure required to maintain a constant flow rate through a needle will vary as the back pressure varies. The back pressure may drop if the needle tip pierces a blood vessel wall and the echogenic fluid is ejected directly into an artery or vein. Therefore, by monitoring the pressure and the rate of change of pressure, the needle may be positioned to deliver therapeutic agents directly into a particular blood vessel.

These claims, and the language used therein, are to be understood in terms of the variants of the invention, which have been described. They are not to be restricted to such variants, but are to be read as covering the full scope of the invention as is implicit within the invention and the disclosure that has been provided herein.

The foregoing has constituted a description of specific embodiments showing how the invention may be applied and put into use. These embodiments are only exemplary. The invention in broader, and more specific aspects, is further described and defined in the claims that now follow.

1-49. (canceled)

50. A medical device used in ultrasonic guided positioning comprising:
a. a fluid container having a discharge end,
b. a fluid discharge device disposed in connection with the fluid container so as to define a fluid retaining reservoir, the discharge device configured to apply a selected pressure to a fluid in the fluid retaining reservoir for ejecting said fluid from the reservoir through the discharge end,
c. a first conduit having an entrance end and an exit end and defining a first passage therebetween, the entrance end disposed at the discharge end of the fluid container, the first passage in communication with the reservoir;
d. a needle having a connector end and a distal tip and defining a needle passage therebetween, the connector end in communication with the exit end of the first conduit, the needle passage in communication with the first passage;
e. a fluid supply operatively connected to the fluid discharge device selectively applying the selected pressure to the fluid,
wherein the selected pressure moves the fluid through the discharge end of the fluid container and travels a first flow path through the first passage and through the needle passage, for ejection of the fluid at the distal tip at a fluid flow rate suitable for detection by ultrasound.

51. The medical device of claim 50, wherein the fluid includes an echogenic fluid.

52. The medical device of claim 50, wherein the fluid includes a therapeutic agent.

53. The medical device of claim 50, wherein the fluid supply comprises a drive mechanism operatively connected to the fluid discharge device and an actuator for the selective operation of the drive mechanism.

54. The medical device of claim 53, wherein the actuator is manually operable, mechanized or programmable.

55. The medical device of claim 52, further comprising a controller electrically connected to the drive mechanism and to the actuator for selectively applying the selected pressure and thereby control the fluid flow rate.

56. The medical device of claim 50, wherein the fluid supply includes means for adjusting the fluid volume of the fluid ejected at the distal tip.

57. The medical device of claim 50, wherein the fluid container is a syringe and the fluid discharge device is a plunger slidably disposed within the syringe.

58. The medical device of claim 50, further comprising a valve member disposed at a selected position on the first conduit, said valve member having a seal for selectively reducing or stopping throughput of the fluid into or within the first passage.

59. The medical device of claim 58, wherein the valve member is disposed at the entrance end of the first conduit.

60. The medical device of claim 58, wherein the valve member is a one-way valve member for permitting fluid flow into the first passage and to prevent fluid flow in the reverse direction into the discharge end of the fluid container.

61. The medical device of claim 50, further comprising an adaptor for releasable coupling of the connector end of the needle to the exit end of the first conduit, the adaptor defining an adaptor passage for maintaining communication between the needle passage with the first passage.

62. The medical device of claim 61, wherein said adaptor includes means for releasably coupling a probe to the needle within the needle passage, the adaptor passage and the needle passage sized to permit the insertion of the probe therein, said probe extending beyond said distal tip.

63. The medical device of claim 62, wherein said probe comprises a therapeutic means.

64. The medical device of claim 63, wherein the therapeutic means includes means for applying at least one of radio frequency, microwave heating, cryosurgical freezing, or brachytherapy.

65. The medical device of claim 54, further comprising:
a. a port connector;
b. a second conduit having a second entrance end and a second exit end and defining a second passage therebetween, the second exit end disposed at the port connector;
c. a second connector disposed at the second entrance end for connection for the second entrance to a selected medical component,
wherein the port connector is disposed at a selected portion of the first conduit or at the valve member for permitting communication between the second passage and the first passage.

66. The medical device of claim 65 wherein the selected medical component includes:
a. a second fluid container having a second discharge end,
b. a second fluid discharge device disposed in connection with the second fluid container so as to define a second fluid retaining reservoir, the second discharge device configured to apply a second selected pressure to a second fluid in the second fluid retaining reservoir for ejecting said second fluid from the second reservoir through the second discharge end,
c. a second fluid supply operatively connected to the second fluid discharge device for selectively applying the second selected pressure to the fluid,
wherein the second selected pressure ejects the second fluid through the second discharge end of the second fluid container and travels a second flow path through the second passage, through to one of the valve member or to the selected portion of the first passage, and through the needle passage, for ejection at the distal tip at a second flow rate.

67. The medical device of claim 66, wherein the second fluid supply comprises a second drive mechanism operatively connected to the second fluid discharge device and a second actuator for the selective operation of the second drive mechanism.

68. The ultrasonically enhanced device of claim 67, wherein the second actuator is manually operable, mechanized or programmable.

69. The medical device of claim 66, further comprising a second transducer for sensing the second selected pressure applied to the second fluid and for outputting an electrical signal reflective of the second selected pressure for input to the controller.

70. The medical device of claim 66, further including a switch configured for switching actuation of the first fluid supply and the second fluid supply.

71. The medical device of claim 70, wherein the second fluid is a therapeutic agent.

72. The medical device of claim 71, wherein the therapeutic agent includes one or more of:
a. a liquid drug,
b. a solid drug suspended in a fluid,
c. a drug eluting microsphere, or other acoustically activated drug delivery system, suspended in a fluid,
d. a radioisotope labeled drug,
e. a radioisotope labeled particle,
f. an imaging system contrast agent for imaging systems including CT scans, MRI, ultrasound or X-ray.

73. The medical device of claim 65, wherein the selected medical component includes a vacuum source for use in tissue aspiration for performing a biopsy.

74. The medical device of claim 65, wherein the selected medical component includes a vacuum source for use in fluid or material drainage.

75. The medical device of claim 65, wherein the medical component includes a catheter for supplying fluids.

76. The medical device of claim 50, further comprising an ultrasound transducer, or multi-transducer array, supported in the housing and in contact with the first conduit in communication with the first passage, the ultrasound transducer or array for transmitting an ultrasound pulse or continuous ultrasound through the needle passage.

77. The medical device of claim 50, further including an adaptor for supporting an ultrasound transducer probe, or multi-transducer probe array, the ultrasound transducer probe or array for transmitting an ultrasound pulse or continuous ultrasound through the needle passage.

78. An ultrasonic guided positioning system for detecting a medical device, comprising:
a. a medical device of claim 50
b. an ultrasound transducer for transmitting and receiving pulses;
c. an ultrasound display; and
d. a system controller electrically connected to each of components (a) to (c), the system controller controlling, detecting or displaying the location of the distal tip of the needle on the ultrasound display.

79. A ultrasonic guided positioning method for detecting a medical device, comprising:
a. dispensing a fluid from a distal tip of a needle of a medical device, the fluid having a selected flow rate for detection by a medical device, the device having:
i. a fluid container having a discharge end,
ii. a fluid discharge device disposed in connection with the fluid container so as to define a fluid retaining reservoir, the discharge device configured to apply a selected pressure to a fluid in the fluid retaining reservoir for ejecting said fluid from the reservoir through the discharge end,
iii. a first conduit having an entrance end and an exit end and defining a first passage therebetween, the entrance end disposed at the discharge end of the fluid container, the first passage in communication with the reservoir;
iv. a needle having a connector end and a distal tip and defining a needle passage therebetween, the connector end in communication with the exit end of the first conduit, the needle passage in communication with the first passage;
v. a fluid supply operatively connected to the fluid discharge device for selectively applying the selected pressure to the fluid, whereby the selected pressure moves the fluid through the discharge end of the fluid container and travels a first flow path through the first passage and through the needle passage, for ejection at the distal tip at a fluid flow rate suitable for detection by ultrasound;
b. transmitting an ultrasonic pulse from an ultrasound transducer;
c. receiving the ultrasound pulse by the ultrasound transducer; and
d. detecting the fluid ejected from the distal tip.
